(19) United States
(12) Patent Application Publication
(10) Pub. No.: US 2019/0083388 A1
(43) Pub. Date: Mar. 21, 2019

(54) SYNTHENTIC TRANSDERMAL CANNABIDIOL FOR THE TREATMENT OF FOCAL EPILEPSY IN ADULTS

(71) Applicant: ZYNERBA PHARMACEUTICALS, INC., Devon, PA (US)

(72) Inventors: Donna Gutterman, Raleigh, NC (US); Terri Sebree, Gladwyne, PA (US); Theodore Smith, Tubac, AZ (US); John Messenheimer, Moncure, NC (US)

(73) Assignee: ZYNERBA PHARMACEUTICALS, INC., Devon, PA (US)

(21) Appl. No.: 16/133,930

(22) Filed: Sep. 18, 2018

Related U.S. Application Data
(60) Provisional application No. 62/660,198, filed on Apr. 19, 2018.
(60) Provisional application No. 62/652,995, filed on Apr. 5, 2018.
(60) Provisional application No. 62/613,160, filed on Jan. 3, 2018.
(60) Provisional application No. 62/593,575, filed on Dec. 1, 2017.
(60) Provisional application No. 62/560,446, filed on Sep. 19, 2017.

(51) Int. Cl. A61K 9/00; A61K 31/047; A61K 9/06; A61P 25/08; A61K 31/4015; A61K 31/55; A61K 31/357; A61K 31/53; A61K 31/165; A61K 31/5513; A61K 31/19; A61K 31/4166

(52) U.S. Cl. CPC A61K 9/0019; A61K 31/047; A61K 9/06; A61K 9/0053; A61P 25/08; A61K 31/4015; A61K 31/4166; A61K 31/357; A61K 31/53; A61K 31/165; A61K 31/5513; A61K 31/19; A61K 31/55

(57) ABSTRACT

The present technology relates to a method of reducing seizure frequency in a subject having epilepsy by transdermally administering an effective amount of cannabidiol (CBD) to the subject wherein the seizure frequency is reduced.

### CROSS REFERENCE TO RELATED APPLICATIONS

This application claims the benefit of and priority to United States Provisional Patent Application No. 62/560,446 filed Sep. 19, 2017; 62/593,575 filed Dec. 1, 2017; 62/613,160 filed Jan. 3, 2018; 62/652,995 filed Apr. 5, 2018; and 62/660,198 filed Apr. 19, 2018. The entire contents of each of which are incorporated herein by reference in their entirety.

### FIELD OF THE TECHNOLOGY

The present disclosure relates to a method of reducing seizure frequency in a subject having epilepsy by transdermally administering an effective amount of cannabidiol (CBD) to the subject wherein the seizure frequency is reduced.

### BACKGROUND

Cannabinoids are a class of chemical compounds found in the Cannabis plant. The two primary cannabinoids contained in Cannabis are cannabidiol, or CBD, and 49-tetrahydrocannabinol, or THC. CBD lacks the psychoactive effects of THC. Studies have shown that CBD can be used to treat disorders such as epilepsy, arthritis, and cancer.

Epilepsy is a disease characterized by an enduring predisposition to generate seizures and by the neurobiological, cognitive, psychological and social consequences of the condition. An epileptic seizure is a transient occurrence of signs and/or symptoms due to abnormal excessive or synchronous neuronal activity in the brain. The seizures in epilepsy may be related to a genetic disorder or a brain injury such as trauma or stroke, but most often the cause is unknown. There are more than 200,000 cases of epilepsy in the United States per year.

Generalized epilepsy affects both hemispheres of the brain from onset. Focal epilepsy (formerly called partial onset seizures) are seizures that affect only one hemisphere or lobe of the brain initially. Symptoms of focal epilepsy vary depending on which hemisphere or lobe of the brain the seizure occurs.

### SUMMARY

The present disclosure relates to a method of reducing seizure frequency in a subject having epilepsy, including transdermally administering an effective amount of cannabidiol (CBD) to the subject wherein the seizure frequency is reduced.

The seizure frequency can be reduced by 25%. In some embodiments, the seizure frequency is reduced by 30%. The seizure frequency can be reduced by 50%. The seizure frequency can be reduced by 65%. The reduction in seizure frequency can be a reduction from a baseline seizure frequency prior to the administration of an effective amount of CBD. In some embodiments, the reduction in seizure frequency is measured by weekly seizure reduction. In some embodiments, the reduction in seizure frequency is measured by seizure frequency per 28 day period. In some embodiments, the reduction in seizure frequency is measured by monthly seizure frequency.

In some embodiments focal onset seizures (formerly known as partial onset seizures) in adults are reduced. An adult is a subject who is eighteen (18) years of age or older. In some embodiments focal aware seizures (formerly known as simple partial seizures) are reduced. Focal impaired awareness seizures (formerly known as complex partial seizures) can be reduced. Focal impaired awareness with generalized tonic-clonic seizures (formerly known as complex partial with generalized tonic-clonic seizures) can be reduced.

The subject can have a high seizure frequency. The epilepsy can be drug resistant epilepsy (formerly known as refractory epilepsy).

In some embodiments, the method also includes administering at least one anti-epileptic drug selected from the group consisting of levetiracetam, carbamazepine, topiramate, lamotrigine, lacosamide, clonazepam, valproate, phenytoin, eslicarbaazepine, clobazam, and oxcarbazepine. Anti-epileptic drugs can be, for example, anticonvulsants. The CBD transdermal gel can be used as an adjunctive therapy with the at least one anti-epileptic drug. In some embodiments, the CBD transdermal gel can be used as an adjunctive therapy with two or three anti-epileptic drugs. The CBD transdermal gel can also be used as a monotherapy.

In some embodiments, the CBD is (−)-CBD. The CBD can be synthetic CBD. The CBD can be pure CBD.

The effective amount of CBD can be between about 195 mg and about 780 mg total daily. The CBD can be administered in a single daily dose. In some embodiments, the CBD is administered in two daily doses.

In some embodiments, the effective amount of CBD is 195 mg in divided daily doses. The effective amount of CBD can be 390 mg in divided daily doses. In some embodiments, the effective amount of CBD is 585 mg in divided daily doses. The effective amount of CBD can be 780 mg in divided daily doses.

The effective amount of CBD can be provided in a 97.5 mg single use sachet. In some embodiments, the effective amount of CBD is provided in a 195 mg single use sachet. The effective amount of CBD can be provided in a 390 mg single use sachet.

The CBD is formulated as a gel. In some embodiments, the CBD is formulated as a permeation enhanced gel. The gel can contain 4.2% (wt/wt) CBD or 7.5% (wt/wt) CBD.

In some embodiments, the transdermal preparation can be a cream, a salve or an ointment. The CBD can be delivered by a bandage, pad or patch.

Transdermally administering an effective amount of CBD can reduce an intensity of at least one adverse event relative to orally administering CBD. The at least one adverse event can be somnolence, psychoactive effects, liver function, GI related adverse events, diarrhea, decreased appetite, fatigue, pyrexia, vomiting, lethargy, upper respiratory tract infection, convulsion, or combinations thereof. In some embodiments, transdermally administering an effective amount of CBD reduces an intensity of at least one adverse event by about 15%, 20%, 25%, 30%, 35%, 40%, 45%, 50%, 55%, 60%, 65%, 70%, 75%, 80%, 85%, 90%, or 95% relative to orally administering CBD.

In some embodiments, the subject is an adult, i.e., eighteen (18) years of age or older.

In some embodiments, the reduction in seizure frequency occurs after three months. The reduction in seizure frequency can occur after twelve (12) weeks. In some embodiments, the reduction in seizure frequency occurs after 6 months. The reduction in seizure frequency can occur after 24 weeks.

The present disclosure relates to a method of reducing seizure frequency in a subject having epilepsy. The method includes transdermally administering a first effective amount of cannabidiol (CBD) to the subject for a time period, wherein the seizure frequency is reduced. The method also includes transdermally administering a second effective amount of CBD to the subject after the time period, wherein the second effective amount of CBD is less than the first effective amount of CBD and wherein the reduced seizure frequency is maintained.

The time period can be twelve (12) weeks. The time period can be twenty four (24) weeks.

### BRIEF DESCRIPTION OF THE DRAWINGS

FIG. 1 is a graph showing the median percent reductions in monthly seizure rates (efficacy population), according to an embodiment of the technology.

FIG. 2 is a graph showing the median percent reductions in seizure rates for patients having focal aware seizures (type B), according to an embodiment of the technology.

FIG. 3 is a graph showing the median percent reductions in seizure rates for patients having focal impaired awareness seizures (type C), according to an embodiment of the technology.

FIG. 4 is a graph showing the median percent reductions in seizure rates for patients having focal impaired awareness with generalized tonic clonic seizures (type D), according to an embodiment of the technology.

FIG. 5 is a graph showing median percent change in seizure rates at week 12 (STAR 1) and Month 3 and 6 (STAR 2), according to an embodiment of the technology.

FIG. 6 is a graph showing the median SF28 over time to each three month interval, according to an embodiment of the technology.

FIG. 7 is a graph showing the median SF28 over time to each three-month interval to 6 months in STAR 2, according to an embodiment of the technology.

FIG. 8 is a graph showing the median SF28 over time for patients without AED changes, according to an embodiment of the technology.

FIG. 9 is a graph showing median change (%) in seizure rates at months 3, 6, 9, and 12 for all CBD transdermal gel treated patients in STAR 2, according to an embodiment of the technology.

FIG. 10 is a graph showing median change (%) in seizure rates at months 3, 6, 9, and 12 for patients treated with placebo or the CBD transdermal gel (195 mg and 390 mg) in STAR 1, according to an embodiment of the technology.

### DETAILED DESCRIPTION

As used herein, the term “treating” or “treatment” refers to mitigating, improving, relieving or alleviating at least one symptom of a condition, disease or disorder in a subject, such as a human, or the improvement of an ascertainable measurement associated with a condition, disease or disorder.

As used herein, the term “clinical efficacy” refers to the ability to produce a desired effect in humans as shown through a Food and Drug Administration (FDA) clinical trial.

As used herein, the term “cannabidiol” or “CBD” refers to cannabidiol; cannabidiol prodrugs; pharmaceutically acceptable derivatives of cannabidiol, including pharmaceutically acceptable salts of cannabidiol, cannabidiol prodrugs, and cannabidiol derivatives. CBD includes, 2-[3-methyl-6-(1-methylethenyl)-2-cyclohexen-1-yl]-5-pentyl-1,3-benzenediol as well as to pharmaceutically acceptable salts, solvates, metabolites (e.g., cutaneous metabolites), and metabolic precursors thereof. The synthesis of CBD is described, for example, in Petilka et al., Helv. Chim. Acta, 52:1102 (1969) and in Mechoulam et al., J. Am. Chem. Soc., 87:3273 (1965), which are hereby incorporated by reference.

As used herein, the term “high seizure frequency” refers to a seizure frequency per 28 day period (SF28) greater than or equal to 15.

As used herein, the term “drug resistant epilepsy” refers to epilepsy where medicine is not adequately treating seizures. Drug resistant epilepsy is a failure of adequate trials of two tolerated, appropriately chosen and used antiepileptic drug schedules (whether as monotherapies or in combination) to achieve sustained seizure freedom.

As used herein, the term “focal onset seizure,” (formerly known as “partial onset seizure”) refers to seizures that affect only one hemisphere or lobe of the brain initially.

As used herein, the term “focal impaired awareness seizure,” (formerly known as “complex partial seizure”) refers to a seizure that affects only one hemisphere or lobe of the brain initially and causes impairment of consciousness

As used herein, the term “focal impaired awareness with generalized tonic-clonic seizure” refers to a focal impaired awareness seizure having characteristics of tonic (stiffening) and clonic (rhythmical jerking).

The term “adjunctive therapy” refers to administration of a therapy to a subject already taking an existing or administration with another therapy, but does not necessarily mean that the therapy is given or provided at the same time or by the same route. For example, the CBD may be administered in addition or adjunct to an existing oral drug therapy.

As used herein, the term “transdermally administering” refers to contacting the CBD with the patient's or subject's skin under conditions effective for the CBD to penetrate the skin.

“Seizure Frequency per 28 day Period” or “SF28” is calculated by the following formula:

SF28=(Total Number of Seizures in D Days)*(28/D)

wherein D is the total number of days for which seizure information is collected for a specific time interval.

The reduction from baseline in seizure frequency (RedSF) during the maintenance period is defined as:

RedSF=SF28(Baseline)−SF28(Maintenance)

Baseline refers to the time period when no CBD gel is administered and the number of seizures during that time period is counted. In other words, the baseline period is a period where the seizure frequency is captured while patients are on their usual AEDs. The baseline period, can be, for example, eight weeks. The subject can have a high seizure frequency during the baseline period. Maintenance refers to the time period when the CBD transdermal gel is administered and the number of seizures in that period is counted. The maintenance period, can be, for example, twelve weeks, three months, six months, nine months, twelve months, fifteen months, eighteen months or twenty months.

The percent reduction from Baseline in seizure frequency during the maintenance period is defined as:

% RedSF=100*[SF28(Baseline)−SF28(Maintenance)/SF28(Baseline)

A patient will be defined as a 50% Responder for a specific treatment period if the patient has a % RedSF≥50%.

In some embodiments, the seizure frequency is calculated per 7 day period to provide a weekly seizure frequency value. The weekly seizure frequency can be reduced by 25%, 30%, 50% or 65%. The reduction in seizure frequency can be a reduction from a baseline seizure frequency prior to the administration of an effective amount of CBD.

The present disclosure relates to a method of reducing seizure frequency in a subject having epilepsy, including transdermally administering an effective amount of cannabidiol (CBD) to the subject wherein the seizure frequency is reduced.

CBD is the primary non-psychoactive cannabinoid found in the Cannabis plant and has low affinity for CB₁ and CB₂ receptors. CBD produces multiple effects, including blocking the equilibrative nucleoside transporter. The orphan G-protein receptor GPR-55, and the transient receptor potential of ankyrin type 1 channel, and regulating the intracellular effects of calcium. The influence of CBD on these targets, each of which is known to play a role in neuronal excitability, is the scientific basis for its antiepileptic potential. The expectation of a wide margin of safety in humans is founded on the results of well-controlled studies in which CBD has exhibited high tolerability across several modes of administration.

Treatment of epilepsy in human patients generally involves antiepileptic drugs and anticonvulsants. Studies have been done that show that CBD can effectively treat Lennox Gastaut and Dravet Syndrome (a type of epilepsy syndrome) but those studies have focused on orally-delivered cannabidiol (CBD) for children with epilepsy.

Transdermal delivery of cannabinoids, e.g., CBD, has benefits over oral dosing because it allows the drug to be absorbed through the skin, directly into the bloodstream. This avoids first-pass liver metabolism, potentially enabling lower dosage levels of active pharmaceutical ingredients with a higher bioavailability (about 25%) and improved safety profile. Transdermal delivery also avoids the gastrointestinal (GI) tract, lessening the opportunity for GI related adverse events and the potential degradation of CBD by gastric acid into THC, which may be associated with unwanted psychoactive and/or euphoric effects. Moreover, oral CBD provides for low (≈6%) and variable bioavailability. Transdermal delivery of CBD can avoid or decrease somnolence adverse events, which are typically present in oral dosing of CBD. Transdermal delivery of CBD can also avoid psychoactive and/or euphoric effects and/or GI related adverse events, which are also typically present in oral dosing of CBD. Transdermal delivery of CBD can avoid liver function adverse events, which are typically present in oral dosing of CBD. In some embodiments, transdermally administering an effective amount of CBD reduces an intensity of at least one adverse event by about 15% to about 95% relative to orally administering CBD.

A clear, transdermal gel was developed to provide consistent, controlled CBD delivery with twice daily (about every 12 hours) dosing. A 4.2% (wt/wt) or 7.5% (wt/wt) CBD gel can be used. The CBD can be in a gel form and can be pharmaceutically-produced as a clear, permeation-enhanced gel that is designed to provide controlled drug delivery transdermally with once- or twice-daily dosing. The CBD gel can be applied topically by the patient or caregiver to the patient's arm, back, leg, or any combination thereof.

Because the zero-order delivery from the transdermal application of the CBD gel can provide a lower Cₘₐₓ than oral or buccal routes of delivery, the CBD transdermal gel usage can result in less systemic exposure, placing it well below the threshold of safety in humans that has been established at higher systemic doses with oral, inhalation and injectable formulations.

It was expected that there would be a zero order kinetics between oral dosing and transdermal dosing, but this was not the case. Without being bound to theory, a two-compartment model with two absorption rates (one faster with lag time and one slower without lag time) was put together because it is believed that transdermal application of CBD gel is applied to the skin, then absorbed through the skin at the site of application, then enters the blood stream, then enters fat tissue all over the subjects body (e.g., liver) as well as the site of action (e.g., the brain). This model was unexpected and new and supports going from a once a day dosing to a twice daily dose, once a steady state level is reached. In addition, ultimately, subjects can have their dosing decreased by up to one half once they have reached a steady state. In addition, the induction period of the CBD transdermal gel is about twelve (12) to twenty four (24) weeks, which may be due to the new and unexpected model of the transdermal dosing.

Because CBD is virtually insoluble in water, ethanol and propylene glycol can be used as solubilizing agents and diethylene glycol monoethyl ether can be used as a permeation enhancer. The alcohol content of the CBD transdermal gel is about 54% (wt/wt).

The CBD gel can include diluents and carriers as well as other conventional excipients, such as wetting agents, preservatives, and suspending and dispersing agents.

The CBD gel can include a solubilizing agent, a permeation enhancer, a solubilizer, antioxidant, bulking agent, thickening agent, and/or a pH modifier. The composition of the CBD gel can be, for example, a. cannabidiol present in an amount of about 0.1% to about 20% (wt/wt) of the composition; b. a lower alcohol having between 1 and 6 carbon atoms present in an amount of about 15% to about 95% (wt/wt) of the composition; c. a first penetration enhancer present in an amount of about 0.1% to about 20% (wt/wt) of the composition; and d. water in a quantity sufficient for the composition to total 100% (wt/wt). Other formulations of the CBD gel can be found in International Publication No. WO 2010/127033, the entire contents of which are incorporated herein by reference.

### Example

This study evaluates the safety and efficacy of CBD transdermal gel as adjunctive therapy for the treatment of adult focal epilepsy. The CBD transdermal gel is a clear, permeation-enhanced gel that is designed to provide controlled drug delivery transdermally with once- or twice-daily dosing. A 4.2% (wt/wt) CBD gel was evaluated in this study.

Methods

A study was done, referred to STAR 1 (Synthetic Transdermal Cannabidiol for the Treatment of Epilepsy), which was a Phase 2, randomized, double-blind, placebo-controlled, study assessing cannabidiol, administered as a transdermal gel BID (twice a day) for twelve (12) weeks to adults with focal epilepsy (Maintenance Period). Following an eight-week baseline (Baseline Period), patients were randomized 1:1:1 to CBD 390 mg daily in divided does (e.g., 195 mg twice daily), CBD 195 mg daily in divided doses (e.g., 97.5 mg twice daily), or placebo. The divided daily doses were given every 12 hours (±2 hours). The CBD transdermal gel and placebo were massaged into both the right and left shoulders and/or upper arms until the area was dry. The primary efficacy endpoint was the change in seizure frequency over the entire treatment period versus baseline. The primary efficacy endpoint is based on the reduction in seizure frequency per 28-day period (SF28) comparing the Baseline Period to the Maintenance Period.

At the end of week 12, patients could elect to roll into an ongoing STAR 2 18-month (amended to 24 month) open label extension study evaluating the CBD transdermal gel at 390 mg in adult patients with focal epilepsy. Presented below is data through nine months of total exposure to the CBD transdermal gel (three months of treatment in STAR 1 and six months of treatment in STAR 2).

Demographics and Baseline Characteristics

Patients (N=188) were randomized into STAR 1. The mean age was 39 (18-71) years. At baseline, patients were taking an average of 2.5 antiepileptic drugs (AEDs) with a median of 10.6 (3-330) seizures monthly. By group, the median monthly seizure frequency at baseline was 10.5 for the placebo group, 14.0 from the 195 mg daily in divided doses group and 10.14 for the 390 mg in divided doses group.

Of the 188 randomized patients, 186 were analyzed for efficacy, and 174 completed the 12-week STAR 1 study. 171 patients (98% of STAR 1 completers) continued into STAR 2. Patients were taking a wide range of AEDs, with a median of 3.0 AEDs. Use of clobazam was excluded in both the STAR 1 and STAR 2 studies because of known interactions between clobazam and CBD.

Efficacy

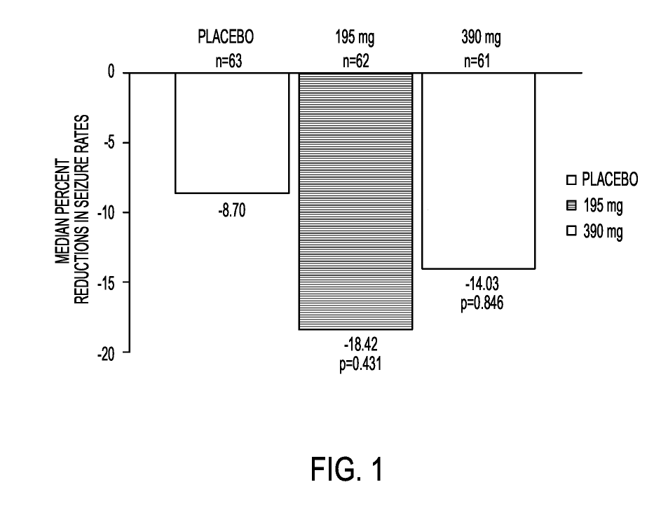

Referring to FIG. 1, after 12 weeks of blinded treatment, the median reduction in focal seizures was 18.42% with the CBD transdermal gel at 195 mg/day (n=62), 14.03% with the CBD transdermal gel at 390 mg/day (n=61), and 8.70% with placebo (n=63). There were no statistically significant differences in efficacy between 195 mg/day (p=0.431), 390 mg/day (p=0.846) and placebo. No secondary endpoint showed a statistically significant difference of 195 mg/day, 390 mg/day and placebo. The 50% responder rate was similar across all treatment groups: placebo=23.8%, 195 mg/day=21% (p=0.414), and 390 mg/day=16.4% (p=0.21).

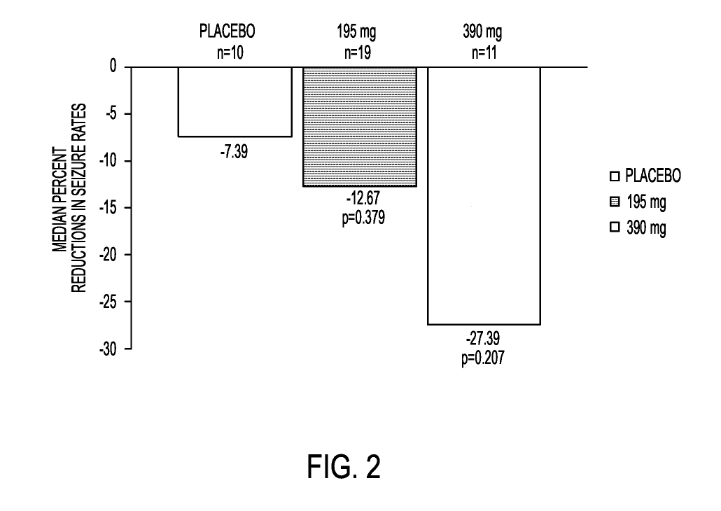

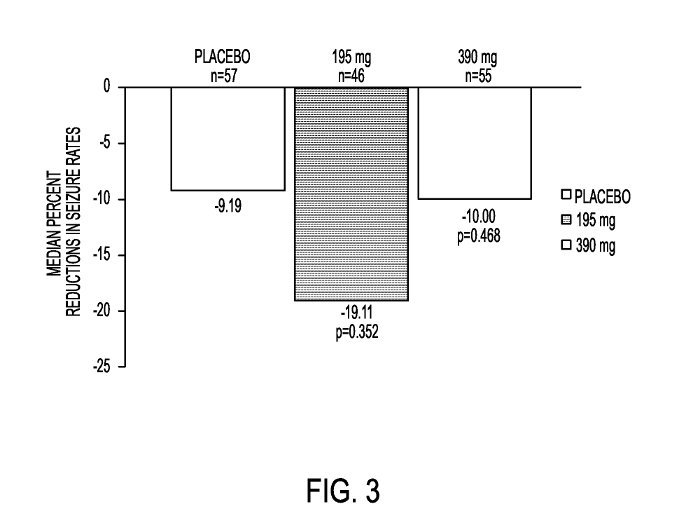

Referring to FIGS. 2 and 3, it can be seen that there was no statistically significant differences in efficacy between 195 mg/day, 390 mg/day and placebo for patients with focal aware (type B) or patients with focal impaired awareness (type C) seizures. What was unexpected and surprising is that there was a near statistically significant difference in efficacy between 195 mg/day (p=0.071) and placebo for patients with focal impaired awareness seizures with generalized tonic clonic seizures. It was also unexpected and surprising that the lower dosage of the CBD transdermal gel resulted in greater efficacy for patients with focal impaired awareness seizures with generalized tonic clonic seizures.

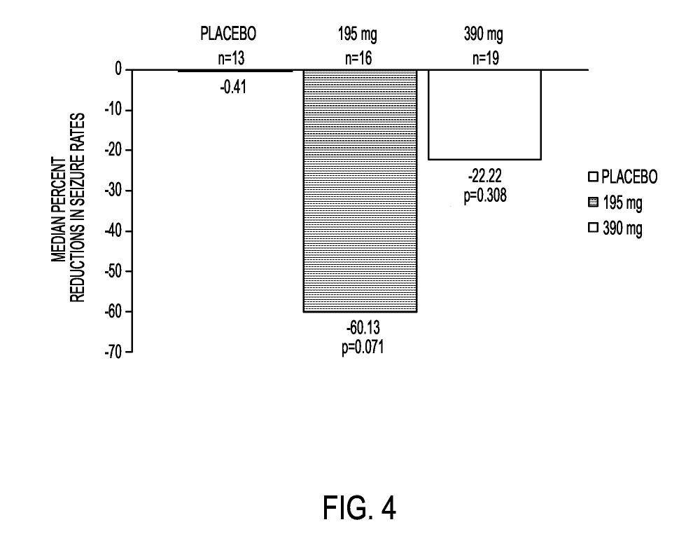

Referring to FIG. 4, of interest are the results from focal impaired awareness seizures with generalized tonic clonic seizures, where 60% median reduction (p=0.071) was observed in the 195 mg/day treatment group, compared to 22.2% median reduction (p=0.308) for the 390 mg/day group and a 0.41% medium reduction for the placebo group. It was unexpected that a lower dose of the CBD transdermal gel would result in a great median reduction for focal impaired awareness seizures with generalized tonic clonic.

The lack of separation of the CBD transdermal gel from placebo in STAR 1 may have been due in part to 15 (24%) placebo-treated patients who achieved at least a 50% reduction in focal seizures; 13 of these 15 patients had a relatively low baseline seizure rate (<15 focal seizures per month). Table 1 shows that the majority of placebo “super” responders (>50%) are female.

TABLE 1

| | Placebo > 50% | |
|---|---|---|
| | Responders (n = 15) | Total Population |
| Median Age (yrs) | 45 | 195 mg = 35; 390 mg = 40 |
| Female (%) | 73% | 195 mg = 49%; 390 mg = 58% |
| Median Baseline | 11 | 10.6 |
| Sz Frequency | | |
| (monthly) | | |
| AED Use | | Mean = 2.5 |
| | | Median = 3 |
| Median Epilepsy | 24 | 21 |
| Duration (yrs) | | |

In addition, placebo responders were more likely taking topiramate than the broad population. Table 2 shows AED used among “super responder” and entire patient population

TABLE 2

| | Placebo > 50% Responder | Total Population |
|---|---|---|
| Anti-Epileptic Drug | (n = 15) | (n = 188) |
| Levetiracetam | 47% | 45% |
| Carbamazepine | 40% | 41% |
| Topiramate | 33% | 16% |
| Lamotrigine | 27% | 32% |
| Lacosamide | 20% | 28% |
| Clonazepam | 20% | 14% |
| Valproate | 13% | 22% |

In STAR 1, patients with high frequency seizures (defined as a baseline seizure frequency of ≥15 per month) taking either the CBD transdermal gel at 195 mg/day or 390 mg/day had a greater percent reduction in seizures compared to patients with high frequency seizures receiving placebo.

171 Patients Rolled into STAR 2.

Post-hoc analysis showed by month three of STAR 2, patients who received the CBD transdermal gel in STAR 1 and STAR 2 (six months total of the CBD transdermal gel) had greater reductions in seizure frequency relative to those who only received the CBD transdermal gel for the three month in STAR 2 (i.e., those who received placebo in STAR 1). See Table 3.

TABLE 3

| | | ZYN002 | |
|---|---|---|---|
| STAR 2 Patients with Data to | | 195 mg* | ZYN002 390 mg* |
| Month 3 | Placebo* | daily | daily |
| STAR 1 Reduction from | 8.7 | 18.42 | 14.03 |
| baseline at Week 12 | (n = 63) | (n = 62) | (n = 61) |
| (Median %) | | | |
| STAR 2 Reduction from | 13.40 | 32.68 | 30.04 |
| STAR 1 baseline at Month 3 | (n = 59) | (n = 55) | (n = 18) |
| (Median %) | | | |

*Assigned treatment in STAR 1. All patients received 390 mg/day CBD transdermal gel in STAR 2.

In addition, the use of CBD transdermal gel for nine months provides better benefit. The efficacy is maintained with continued treatment. Patients taking placebo in STAR 1 and completing six months in STAR 2 had less seizure reduction than patients taking the CBD transdermal gel (195 mg or 390 mg) during STAR 1. See Table 4.

TABLE 4

| | | | ZYN002 |
|---|---|---|---|
| | | | 390 mg* |
| STAR 2 Patients with Data to | Placebo* | ZYN002 195 mg* | daily |
| Month 6 | (N = 24) | daily (N = 21) | (N = 18) |
| STAR 1 Reduction from | 7.68 | 33.33 | 18.48 |
| baseline at Week 12 | | | |
| (Median %) | | | |
| STAR 2 Reduction from | 26.08 | 58.06 | 47.90 |
| STAR 1 baseline at Month 3 | | | |
| (Median %) | | | |
| STAR 2 Reduction from | 26.53 | 65.23 | 48.45 |
| STAR 1 baseline at Month 6 | | | |
| (Median %) | | | |

*Assigned treatment in STAR 1. All patients received 390 mg/day CBD transdermal gel in STAR 2.

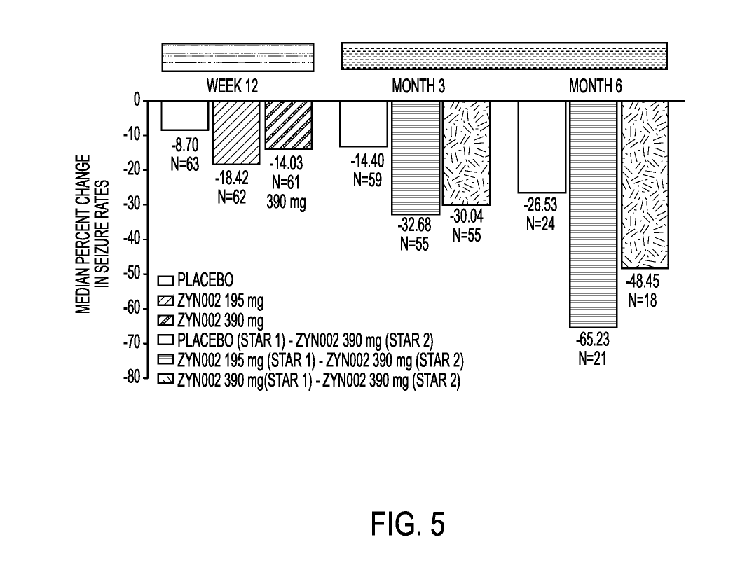

Continued exposure to CBD transdermal gel in STAR 2 (all patients dosed with 390 mg/day) resulted in clinically meaningful reductions in seizures. Patients taking CBD transdermal gel for six months (three months during STAR 1 and three months during STAR 2) experienced a greater than 30% median reduction in seizures from baseline. Patients taking CBD transdermal gel for nine months (three months during STAR 1 and six months during STAR 2) experienced a greater than 65% (195 mg/day in STAR 1 and 390 mg/day in STAR 2) and a greater than 48% (390 mg/day in STAR 1 and STAR 2) median reduction in seizures from baseline. The results are summarized in Table 4 above and in FIG. 5.

Seizure control was evaluated as a function of duration on the CBD transdermal gel, regardless of initial randomization group or dose. Longer exposure to the CBD transdermal gel resulted in greater improvements in seizure frequency, with median percent change in seizures from −16.3% at 3 months (n=170), to −27.3% at 6 months (n=148), −50.2% at 9 months (n=98), and −58.0% at 12 months (n=70).

As shown in Table 5, patients who were administered the CBD transdermal gel for six months had the best response

TABLE 5

| | PBO/ | 195 mg/ | |
|---|---|---|---|
| | 390 mg* | 390 mg* | 390 mg/390 mg* |
| Patients in STAR 1 with | 32% | 41% | 29% |
| ≥30% reduction at week 12 | (n = 60) | (n = 56) | (n = 55) |
| (patient number) | | | |
| Patients from STAR 1 with | 36% | 53% | 51% |
| ≥30% reduction in STAR 2 at | (n = 59) | (n = 55) | (n-55) |
| month 3 | | | |
| Patients from STAR 1 with | 42% | 81% | 78% |
| ≥30% reduction in STAR 2 at | (n = 24) | (n = 21) | (n = 18) |
| month 6 | | | |

*Assigned treatment in STAR 1. All patients received 390 mg/day CBD transdermal gel in STAR 2.

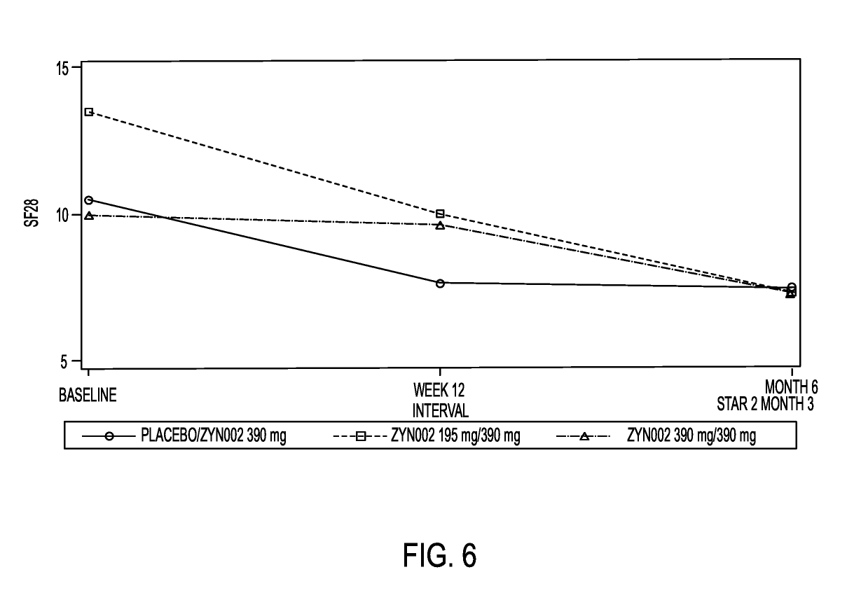

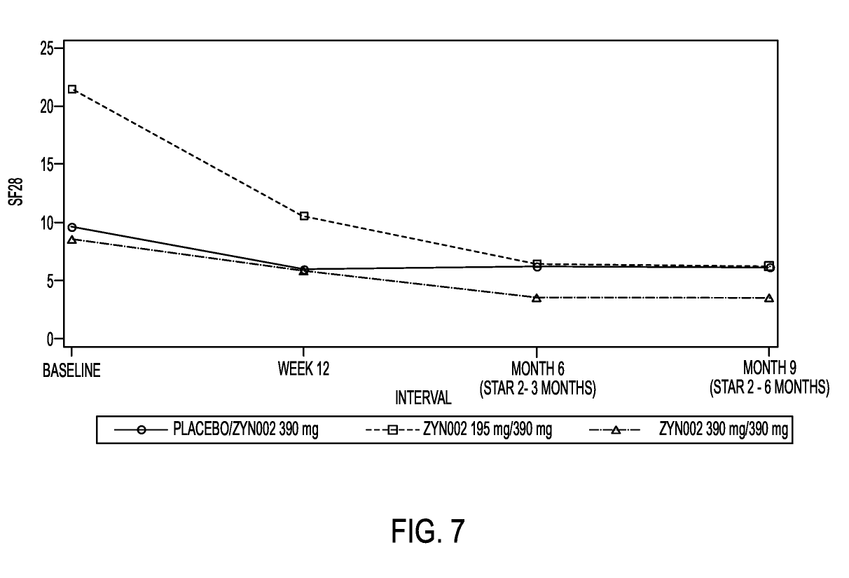

These results can also be seen in FIG. 6, which shows Median SF28 over time to each three-month interval. As can be seen from FIG. 6, the two non-placebo groups had continued improvement with longer exposure. FIG. 7 shows median SF28 over time to each three-month interval to six months in STAR 2. The continued improvement or maintained reduction in seizure frequency can be seen for the two non-placebo groups.

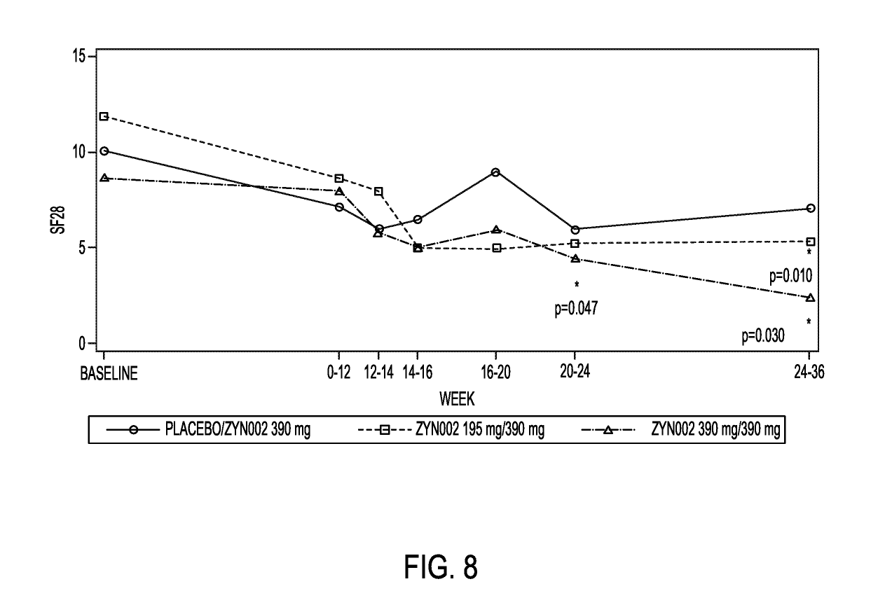

FIG. 8 shows the median SF28 over time for patients who did not have any AED changes. The graph of FIG. 8 shows that with more cumulative CBD transdermal gel, the greater the efficacy with statistically significant results.

Post-hoc analysis showed that placebo responders are likely to be women and the 390 mg/day non-responders have the highest CBD plasma concentration (although this was highly variable). Table 6 shows percent reduction from placebo from baseline to treatment in seizure rates (SF28) by demography.

TABLE 6

| | | Placebo | | 195 mg | | 390 mg |
|---|---|---|---|---|---|---|
| | Placebo | Non- | 195 mg | Non- | 390 mg | Non- |
| | Responder | Responder | Responder | Responder | Responder | Responder |
| | (n = 22) | (n = 41) | (n = 26) | (n = 36) | (n = 24) | (n = 37) |
| Median Age (yr.) | 42 | 37 | 33 | 38 | 37 | 41 |
| Female (%) | 64 | 54 | 46 | 50 | 58 | 57 |
| Median Weight (kg) | 70 | 72 | 80 | 72 | 71 | 75 |
| Median BMI (kg/m²) | 24 | 25 | 27 | 25 | 26 | 24 |
| Median Txt | 84 | 84 | 84 | 84 | 84 | 84 |
| Duration (days) | | | | | | |
| Week 12 Median CBD | NA | NA | 3.67 | 4.89 | 5.81 | 8.65 |
| concentration (ng/ml) | | | | | | |

Analysis also showed that the 26- to 40-year-old patients had the best response, with youngest and oldest patients getting nominally worse. Table 7 shows the percent reduction from placebo from baseline to treatment in seizure rates (SF28) by age.

TABLE 7

| Age Groups and Etiology | 195 mg/day | 390 mg/day |
|---|---|---|
| 18-25 years of age | −9.03%, NS | −28.83%, NS |
| | (n = 14) | (n = 10) |
| 26-40 years of age | 29.50% (p = 0.036) | 6.06%, NS |
| | (n = 23) | (n = 21) |
| >40 years of age | −32.05% (p = 0.092) | −32.55% (p = 0.077) |
| | (n = 25) | (n = 30) |

Analysis further showed that only males at the 195 mg/day dose had a positive response, while females at both doses had poor responses. Table 8 shows the percent reduction from placebo from baseline to treatment in seizure rates (SF28) by gender.

TABLE 8

| | | |
|---|---|---|
| Females | 195 mg/day | 390 mg/day |
| | (n = 30) | (n = 35) |
| | −1.7%, NS | −6.5%, NS |
| Males | 195 mg/day | 390 mg/day |
| | (n = 32) | (n = 22) |
| | 7.1%, NS | −12.4%, NS |

In STAR 1, adverse event rates of CBD transdermal gel 195 mg/day were 49.2%; the adverse event rates of CBD transdermal gel 390 mg/day were 51.6%; and the adverse event rates of placebo were 41.3%. The most common adverse events were upper respiratory tract infection (viral and bacterial; 16%), headache (11%), fatigue (7%), and laceration (5%). The two-treatment-emergent adverse events that occurred in >5% of CBD transdermal gel patient and greater than the placebo were fatigue (5.6% for CBD transdermal gel; 1.6% for placebo) and headache (5.6% for CBD transdermal gel; 3.2% for placebo). The plasma levels of CBD were dose-proportional, but there was no correlation between plasma levels and efficacy.

Although the median plasma concentration at four weeks amount 25% responders between four and eight weeks were lower compared to non-responders, the different was nominal. There was no difference in median concentration at eight weeks between responders and non-responders between week eight and twelve. While Cₘₐₓ in a study involving 252 mg BID dose in four subject was higher than the simulated Cₘₐₓ (PopPK model), both demonstrate equal probability of representing the true Cₘₐₓ. The inter-subject pharmacokinetic variability of the CBD transdermal gel is within the expected range for transdermal products.

A small number of patients in STAR 2 had an increase in their background AEDs. The improvements in seizure frequency observed in STAR 2 were not due to these changes in background AEDs. This can be seen by a comparison of FIG. 8, which shows response among those patients who did not have and AED change with FIG. 7 which includes all patients. As can be seen, these two figures are very similar.

Safety

The CBD transdermal gel was very well tolerated with an incidence of adverse events comparable to placebo and no clinically significant differences between the active treatment groups. The safety profile of the CBD transdermal gel was consistent with data from Phase 1 and Phase 2 trials. There were no clinically significant changes in ECGs or laboratory results in patients receiving the CBD transdermal gel. In addition, the CBD transdermal gel had good skin tolerability, with minimal skin erythema.

Summary of Findings

Clinically meaningful responses to CBD transdermal gel, as measured by reductions in focal seizures from the baseline period of STAR 1, are correlated with continued treatment with the CBD transdermal gel. Patients who received the CBD transdermal gel (195 mg/day during STAR 1 for three months and 390 mg/day for six months in STAR 2) for a total of nine months achieved a median reduction in seizures of 65%. Patients who received the CBD transdermal gel (390 mg/day for three months in STAR 1 and six months in STAR 2) achieved a 48% median reduction in seizures from baseline. In addition, the CBD transdermal gel was shown to be very well tolerated through nine months of exposure.

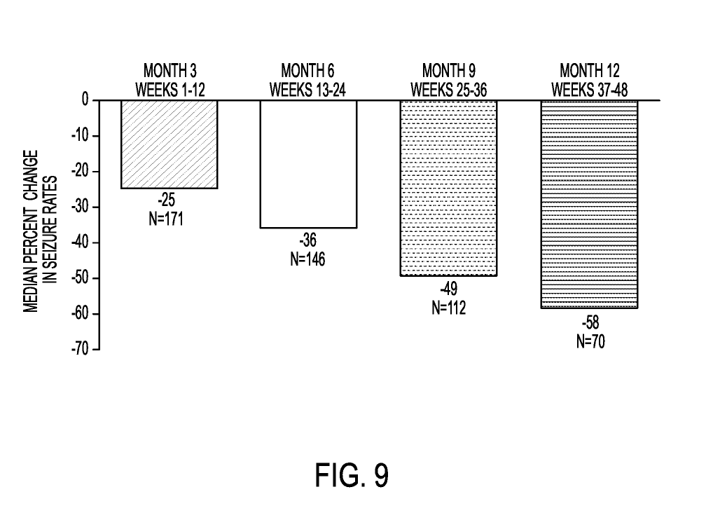

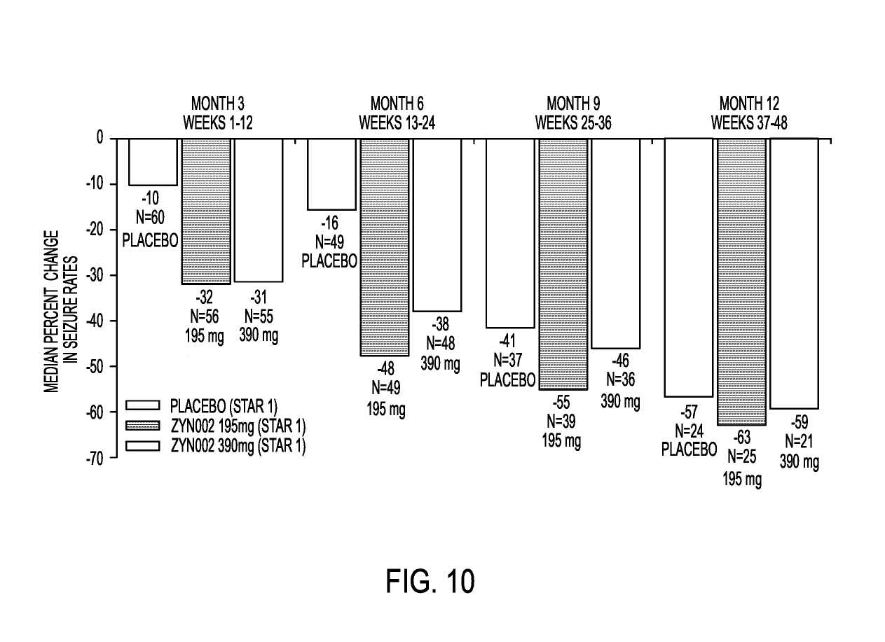

It was found that correlation of continued treatment with the CBD transdermal gel and the reduction in focal seizures from the baseline period of STAR 1 continued through month twelve. FIG. 9 shows the median change (%) in seizure rates at months three, six, nine, and twelve for all patients treated with the CBD transdermal gel in STAR 2. FIG. 10 shows the median change (%) in seizure rates at months three, six, nine, and twelve for patients treated with placebo of the CBD transdermal gel (195 mg and 390 mg) in STAR1. As can be seen in FIGS. 9 and 10, longer exposure to the CBD transdermal gel resulted in greater improvements in seizure frequency among all CBD transdermal gel patient (FIG. 9), including when examined by originally randomized CBD does in STAR 1 (FIG. 10).

The data demonstrates that focal seizures in adults may be effectively treated by a transdermal gel delivery of pharmaceutically-produced cannabidiol. In this population of patients, continued treatment with the CBD transdermal gel was shown to significantly reduce seizure rates compared to baseline. Importantly, baseline seizure frequency appears to be an important indicator of response. The data demonstrates that the CBD transdermal gel can have an effect on focal seizures in adults suffering from drug resistant epilepsy. The potential for a CBD-based treatment with an optimal tolerability profile would be significant for these patients.

STAR 2 Trial Amended

The protocol of the STAR 2 clinical trial was amended to enable the titration of various doses of the CBD transdermal gel based on the observed outcome. The new protocol allows doctors to prescribe the CBD transdermal gel at 195 mg/day, 390 mg/day, 585 mg/day or 780 mg/day. The amended protocol allows doctors to titrate the dose of the CBD transdermal gel up or down.

In the amended STAR 2 trial, all patients will start with a CBD transdermal gel at 195 mg every 12 hours (±2 hours) (390 mg/day), with the option after the first month to either increase or reduce the dose of the CBD transdermal gel. The dose can be increased to 292.5 mg every 12 hours (±2 hours) (585 mg/day). After one month at the 585 mg/day dose, the dose can be increased again to 390 mg every 12 hours (±2 hours) (780 mg/day).

After one month, the CBD transdermal gel dosage can be reduced to 97.5 mg per 12 hours (±2 hours) (195 mg/day). For patients taking the 195 mg daily dose, there is an option to raise the dose back to 390 mg/day and up to a maximum of 585 mg/day.

Each time the dose of the CBD transdermal gel is increased or decreased, the patient will remain on that does for one month to achieve steady state before the dose is changed again.

What is claimed is:

1. A method of reducing seizure frequency in a subject having epilepsy, the method comprising:
transdermally administering an effective amount of cannabidiol (CBD) to the subject wherein the seizure frequency is reduced.

2. The method of claim 1, wherein the seizure frequency is reduced by 30%.

3. The method of claim 1, wherein the seizure frequency is reduced by 50%.

4. The method of claim 1, wherein focal onset seizures in adults are reduced.

5. The method of claim 1, wherein focal aware seizures are reduced.

6. The method of claim 1, wherein focal impaired awareness seizures are reduced.

7. The method of claim 1, wherein focal compared awareness with generalized tonic-clonic seizures are reduced.

8. The method of claim 1, wherein the subject has a high seizure frequency.

9. The method of claim 1, wherein the epilepsy is drug resistant epilepsy.

10. The method of claim 1, administering at least one anti-epileptic drug selected from the group consisting of levetiracetam, carbamazepine, topiramate, lamotrigine, lacosamide, clonazepam, valproate, clobazam, phenytoin, eslicarbaazepine, and oxcarbazepine.

11. The method of claim 1, wherein the CBD is (−)-CBD.

12. The method of claim 1, wherein the effective amount of CBD is between about 195 mg and about 780 mg total daily.

13. The method of claim 1, wherein the effective amount of CBD is 195 mg in divided daily doses.

14. The method of claim 1, wherein the effective amount of CBD is 390 mg in divided daily doses.

15. The method of claim 1, wherein the effective amount of CBD is 585 mg in divided daily doses.

16. The method of claim 1, wherein the effective amount of CBD is 780 mg in divided daily doses.

17. The method of claim 1, wherein the effective amount of CBD is provided in a 97.5 mg single use sachet.

18. The method of claim 1, wherein the effective amount of CBD is provided in a 195 mg single use sachet.

19. The method of claim 1, wherein the effective amount of CBD is provided in a 390 mg single use sachet.

20. The method of claim 1, wherein the CBD is formulated as a gel.

21. The method of claim 20, wherein the CBD is formulated as a permeation enhanced gel.

22. The method of claim 1, wherein the CBD is administered in a single daily dose.

23. The method of claim 1, wherein the CBD is administered in two daily doses.

24. The method of claim 1, wherein the CBD is a synthetic CBD.

25. The method of claim 1, wherein the CBD is a pure CBD.

26. The method of claim 1, wherein transdermally administering an effective amount of CBD reduces an intensity of at least one adverse event relative to orally administering CBD.

27. The method of claim 26, wherein the at least one adverse event is selected from the group consisting of somnolence, psychoactive effects, liver function, and GI related adverse events.

28. The method of claim 1, wherein the subject is an adult.
